(19) United States
(12) Patent Application Publication
(10) Pub. No.: US 2026/0294254 A1
(43) Pub. Date: Oct. 1, 2026

(54) SYSTEMS AND METHODS FOR POSTPARTUM HEMORRHAGE RISK PREDICTION USING MATERNAL HEART RATE

(71) Applicants: VANDERBILT UNIVERSITY, Nashville, TN (US); The Trustees Of The University Of Pennsylvania, Philadelphia, PA (US)

(72) Inventors: James Weimer, Nashville, TN (US); Stefanie Modri, Bridgeton, NJ (US)

(21) Appl. No.: 19/489,015

(22) PCT Filed: May 31, 2024
(86) PCT No.: PCT/US2024/032064

§ 371 Date: Dec. 1, 2025

Related U.S. Application Data
(60) Provisional application No. 63/505,493, filed on Jun. 1, 2023.
(60) Provisional application No. 63/632,194, filed on Apr. 10, 2024.

(51) Int. Cl. A61B 5/02; A61B 5/00; A61B 5/024; A61B 5/0245; A61B 5/11

(52) U.S. Cl. CPC A61B 5/02042; A61B 5/02416; A61B 5/0245; A61B 5/1102; A61B 5/4325; A61B 5/681; A61B 5/7275; A61B 5/7405; A61B 5/742; A61B 5/7455; A61B 5/746; A61B 2560/0462

(57) ABSTRACT

Various examples are provided related to postpartum hemorrhage (PPH) risk prediction. In one example, a PPH risk prediction system includes one or more pulse waveform sensors that can collect heart beat-to-beat timing information of an individual in real time; and processing circuitry that can process the heart beat-to-beat timing information to monitor for previously learned signatures predictive of uterine atony risk and provide a clinical alert notification in response to the monitoring. The notification can be provided through an audible, haptic, dashboard or mobile device. In another example, a method includes obtaining, via one or more pulse waveform sensors, heart beat-to-beat timing information of an individual in real time; processing the heart beat-to-beat timing information to monitor for previously learned signatures predictive of uterine atony risk; and providing a clinical alert notification in response to the monitoring through an audible, haptic, dashboard or mobile device.

### CROSS REFERENCE TO RELATED APPLICATIONS

This application claims priority to, and the benefit of, U.S. provisional applications entitled “Systems and Methods for Postpartum Hemorrhage Risk Prediction Using Maternal Heart Rate” having Ser. No. 63/505,493, filed Jun. 1, 2023, and Ser. No. 63/632,194, filed Apr. 10, 2024, both of which are hereby incorporated by reference in their entireties.

### STATEMENT REGARDING FEDERALLY SPONSORED RESEARCH OR DEVELOPMENT

This invention was made with government support under grant/contract number 1R41HL164191-01 awarded by the National Institutes of Health and grant/contract number 1915398 awarded by the National Science Foundation. The government has certain rights in the invention.

### BACKGROUND

Postpartum hemorrhage (PPH) is a leading cause of maternal mortality. Uterine atony accounts for most (80%) of PPH, where myometrial fatigue is a significant factor that is not directly measurable. Delays in obtaining quality, evidence-based care for postpartum hemorrhage (PPH) are major contributors to making postpartum hemorrhage (PPH) the leading cause of maternal mortality worldwide. While risk screening tools have been developed to predict PPH, the results from the use of these tools have not been robust.

### SUMMARY

Aspects of the present disclosure are related to postpartum hemorrhage (PPH) risk prediction. In one aspect, among others, a PPH risk prediction system, comprises one or more pulse waveform sensors configured to collect heart beat-to-beat timing information of an individual in real time; and processing circuitry configured to: process the heart beat-to-beat timing information to monitor for previously learned signatures predictive of uterine atony risk; and provide a clinical alert notification in response to the monitoring, the notification provided through an audible, haptic, dashboard or mobile device. In one or more aspects, the heart beat-to-beat timing information can be collected from photoplethsymgraphy, electrocardiogram, ballistography, or electromyarthapy. A bed-side or wearable medical device can comprise the one or more pulse waveform sensors. The wearable medical device can be a smart watch. The collected heart beat-to-beat timing information can be stored in memory on the wearable medical device. In various aspects, the processing circuitry can be locally or remotely located. The remotely located processing circuitry can comprise at least one server computer in a cloud-based environment. The remotely located processing circuitry can receive the collected heart beat-to-beat timing information from the one or more pulse waveform sensors. The collected heart beat-to-beat timing information can be stored in memory on the at least one server computer.

In another aspect, a method for PPH risk prediction comprises obtaining, via one or more pulse waveform sensors, heart beat-to-beat timing information of an individual in real time; and processing, by processing circuitry, the heart beat-to-beat timing information to monitor for previously learned signatures predictive of uterine atony risk; and providing, by the processing circuitry, a clinical alert notification in response to the monitoring, the notification provided through an audible, haptic, dashboard or mobile device. In one or more aspects, the heart beat-to-beat timing information can be collected from photoplethsymgraphy, electrocardiogram, ballistography, or electromyarthapy. The heart beat-to-beat timing information can be collected via a bed-side or wearable medical device comprising the one or more pulse waveform sensors. The wearable medical device can be a smart watch. In various aspects, the heart beat-to-beat timing information can be communicated from the one or more pulse waveform sensors to the processing circuitry for processing. The heart beat-to-beat timing information can be communicated to the processing circuitry via a wireless connection. The processing circuitry can be remotely located. The remotely located processing circuitry can comprise at least one server computer in a cloud-based environment. The communicated heart beat-to-beat timing information can be stored in memory on the at least one server computer.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of the architecture of a PPH system, in accordance with various embodiments of the present disclosure.

FIG. 2 illustrates an example of a postpartum hemorrhage (PPH) risk prediction (or predictor) algorithm, in accordance with various embodiments of the present disclosure.

FIG. 3 is a table illustrating clinical and demographic characteristics of derivation and validation cohorts, in accordance with various embodiments of the present disclosure.

FIGS. 4A and 4B includes tables illustrating examples of performance of the PPH risk prediction (or predictor) algorithm, in accordance with various embodiments of the present disclosure.

FIGS. 5A and 5B illustrates examples of pre-birth sensitivity of the PPH risk prediction algorithm compared with admission risk scoring, in accordance with various embodiments of the present disclosure.

FIG. 6 is a schematic diagram illustrating an example of processing circuitry that can be used to implement the PPH risk prediction methodology, in accordance with various embodiments of the present disclosure.

### DETAILED DESCRIPTION

Disclosed herein are various examples related to postpartum hemorrhage (PPH) risk prediction. Reference will now be made in detail to the description of the embodiments as illustrated in the drawings, wherein like reference numbers indicate like parts throughout the several views.

The risk factors contained in risk screening tools, and their stratification, were initially based on expert opinion only, though validation studies and subsequent revisions to the tools themselves have been published since the California Maternal Quality Care Collaborative (CMQCC) published the first tool in 2010. Consistent shortcomings of these tools are their low-to-moderate specificity and sensitivity; positive predictive values of <10%, and more than 40% of hemorrhages occurring in patients designated as low risk in 4 out of 5 reviewed studies. To add, the surveys are performed at static times during the labor process and do not incorporate any patient biometric data. Considering these poor predictive values, a number of approaches have been actively explored to improve the existing models of risk assessment, including the incorporation of intrapartum factors or non-obstetric risk factors.

The most common cause of postpartum hemorrhage (PPH) is uterine atony, where myometrial fatigue is a significant contributing factor. While minimally invasive observation of myometrial fatigue is not clinically available, it is posited that specific variations in maternal heart beat-to-beat timing during active labor contractions can be modeled to correlate with increasing myometrial fatigue. This is based on the physiological fact that during contractions, blood is forced from the uterus and into the maternal circulatory system, resulting in variations in maternal heart beat-to-beat and peripheral blood volume. This fact can be combined with an assumption that the changes in maternal heart beat-to-beat due to contractions would vary based on myometrial fatigue. Consequently, it is hypothesized that wearable photoplethysmography (PPG) data contains rich maternal heart beat-to-beat timing information that can be leveraged to continuously monitor PPH risk during active labor. An automated real-time system for PPH risk during active labor can facilitate better resource planning and clinical care team coordination.

A novel system has been developed that can non-invasively predict PPH more accurately than the state-of-the-art prior to delivery, and a PPH risk prediction (or predictor) algorithm incorporating features from PPG data to provide real-time PPH risk assessments during active labor was developed and validated. Currently, there is a significant effort to provide early prediction to enable proactive clinical planning, and existing solutions rely on PPH detection rather than PPH prediction.

Existing approaches rely on traditional methods, such as blood pressure and pulse beat-to-beat indicators that often yield information too late, or checking sanitary pads after birth, which is old-fashioned and dangerous. Current PPH solutions only work towards what to do after PPH has been detected, which is often too late, resulting in death or significant morbidity.

The present system leverages maternal heart beat-to-beat timing information collected by several existing devices in the labor and delivery unit. The system essentially creates a new vital sign that provides a surrogate measure of uterine fatigue that is suspected of being a strong predictor of uterine atony—which accounts for over 80% of all PPH.

This new vital sign utilizes waveform data collected at >5 Hz from a wearable or minimally invasive monitoring device (e.g., pulse oximeter, ECG, maternal fetal monitor). The device performs local pre-processing and filtering of the data. In coordination with a cloud-based backend, the wearable device and algorithms in the cloud will further process the data to predict whether a life-threatening postpartum hemorrhage is likely to occur. An alerting mechanism integrated with the hospital EHR will notify clinical staff to the situation, with the prediction system providing alerts typically between 1 and 6 hours prior to delivery. This gives clinicians ample time to prepare medications, equipment, and personnel to address the potential PPH event. An overview of the system operation is described below.

The system:
(i) performs real-time collection of individual heart beat-to-beat timing information from photoplethsymgraphy, electrocardiogram, ballistography, and/or electromyarthapy;
(ii) stores the collected data in a medical device or cloud server;
(iii) processes the heart beat-to-beat timing stream, either locally to a medical device or in the cloud, to monitor for previously learned signatures that are predictive of uterine atony risk; and
(iv) provides clinical alerting capabilities through audible, haptic, dashboards, and/or mobile devices.

The current performance of the system (based on a 525 patient IRB-approved observational study) is 60%-80% sensitivity at correctly predicting PPH interventions with a positive predictive value (PPV) >50%. For comparison, the current state-of-the-art based on admission risk criteria for predicting PPH provides a 20% sensitivity and 50% PPV. Thus, with minimal hardware changes to the current labor and delivery unit landscape, and with no decrease in positive predictive value, the proposed system will catch at least 3× more PPH that the current state-of-the-art.

The benefits of using the present system can include:
Significant hospital and insurance costs savings associated with morbidity and mortality;
Saving the lives of mothers, especially Black and brown women or birthing people, who are dying at rates 2-4× greater than white women or birthing people;
The ability to work with devices that will measure multiple vital signs wirelessly; and/or
To be another set of eyes and ears for the health care providers to alert them to a hemorrhage.

### System Description

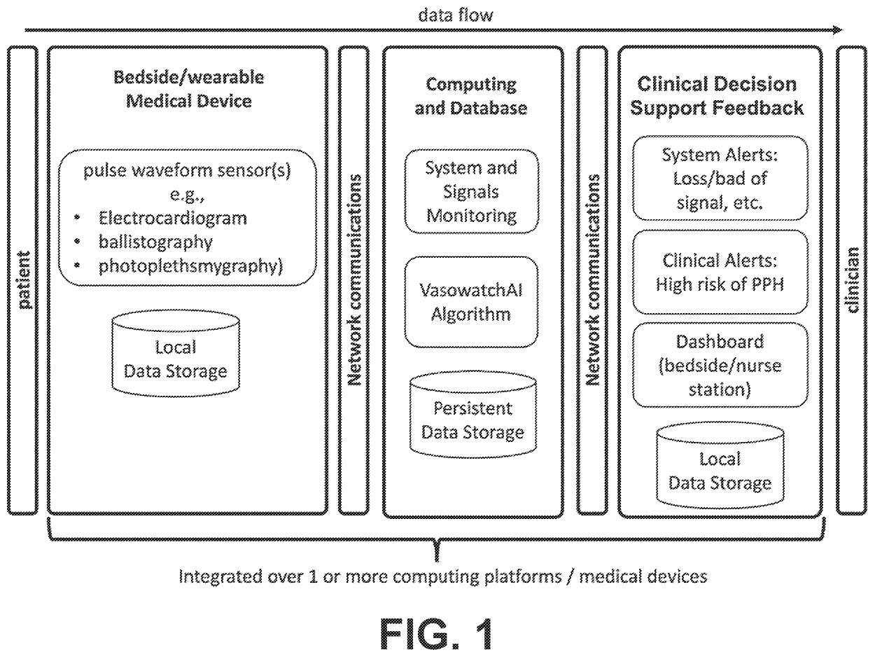

An example of the system architecture is illustrated in FIG. 1. Multiple bed-side and wearable medical devices can be connected to a woman/women or birthing person/people during labor and delivery. A subset (e.g., a single device) contain one or more pulse waveform sensors that produce a timestamped waveform containing heart beat-to-beat timing information. Such waveforms include electrocardiograms, ballistography, and photoplethsymgraphy produced by commonly available medical devices including pulse oximeters and maternal/fetal monitors. The bedside and/or wearable devices can contain local storage to buffer data prior to data transmission via a network communication medium (e.g., IEEE 802.11, IEEE 802.15.4, Bluetooth, near-field communication, serial, or other suitable protocols) to a computing and persistent storage database that may be contained in the device that contains the pulse waveform sensor(s).

Besides persistent data storage, the computing component can contain information processing capabilities for (i) system and signals monitoring and (ii) the PPH risk predictor algorithm. The systems and signals monitoring component processes in real-time the pulse waveform data to assess, using standard techniques, loss of signal (i.e., absence of pulse waveform data), data integrity (i.e., quality of pulse waveform data), and data timeliness (i.e., data transmission delays). When the pulse waveform data assessment violates historical and minimum limits, system alerts are generated and transmitted to the clinical decision support feedback component.

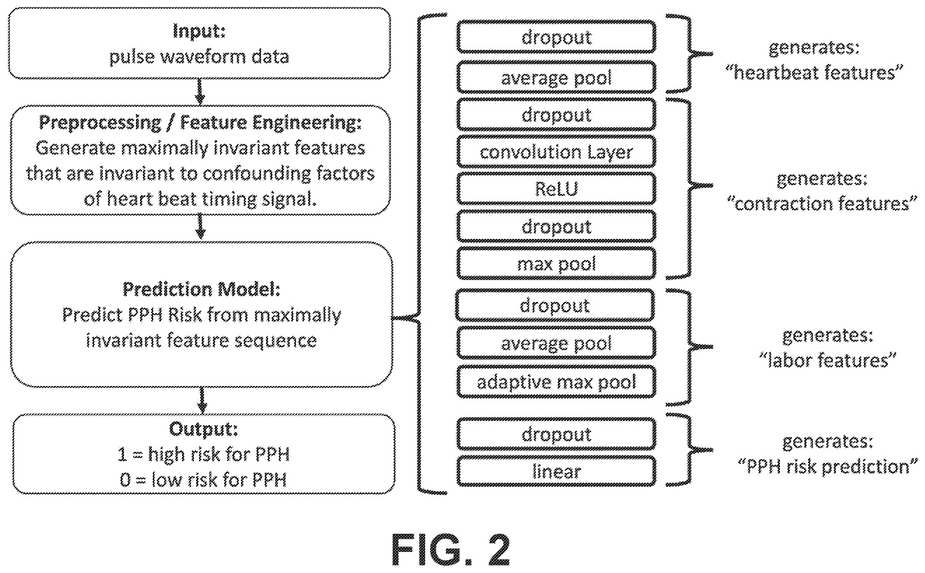

FIG. 2 illustrates an example of the architecture of the PPH risk predictor. Similar to the systems and signals monitoring component, the PPH risk predictor algorithm, as illustrated in FIG. 2, processes pulse waveform data to produce estimates of the PPH risk. The PPH risk can be estimated by first pre-processing the pulse waveform data to produce maximally invariant estimates of RR intervals. The RR interval is the time between two successive R waves and is commonly averaged over multiple heart beats to estimate heart rate. In the PPH risk predictor algorithm, the aim is to extract beat-to-beat timing information in a manner that is maximally invariant to known confounding factors (i.e., nuisance signals). For instance, recognizing that optical pulse waveform sensors that produce photoplethsymgraphy data are affected by skin color, the PPH risk predictor algorithm can extract RR interval estimates by processing the pulse waveform to provide maximal invariance to relative magnitude of the photoplethsymgraphy data. Similar techniques can be applied for other sensor domains, where all seek to provide an RR interval estimate that is maximally invariant to nuisance signals. Once the pulse waveform data is pre-processed into a maximally invariant heart beat-to-beat sequence/stream prediction can be performed leveraging a learned model.

FIG. 2 illustrates the architecture of the learned model that was trained on half the observational study data and evaluated on the remainder. The algorithm architecture of FIG. 2 is structured to contain 4 stages that capture, through machine learning, physiological dynamics present in the maximally invariant heart beat-to-beat sequence/stream in the form of features known to be affected by uterine fatigue that may lead to uterine atony and postpartum hemorrhage. The first stage seeks to produce heart beat-to-beat features, leveraging dropout and average pooling, a new heart beat-to-beat feature is generated every N beats with a stride of N where N is a hyperparameter chosen through validation analysis on the observational study data. These features represent a local average heart beat-to-beat timing based on a number of consecutive beats.

The second stages take the heart beat-to-beat features as input and applies dropout and a convolutional layer consisting of J filters each with a kernel size of K, whose parameters are learned through training and the hyperparameters (i.e., J and K) are chosen through validation analysis. The output of the convolution layer is fed to an activation function (e.g., a rectified linear unit), applies dropout again, then performs max pooling over a window of M convolutional layer outputs at a stride of 1 resulting in contraction features. The intuition behind the contraction feature generator is that while all women or birthing people have differences in contractions, for all women or birthing people contractions are periodic during labor lasting 30 to 90 seconds and occur every 3 to 5 minutes during active labor and delivery.

During training, the contraction feature generator learns patterns of the heart beat-to-beat features over multiple consecutive contractions, and then at runtime applies these patterns as signatures. It is hypothesized, that some signatures are activated when the heart beat-to-beat features over multiple consecutive contractions are aligned with a uterus that is not fatigued, while others are activated by a fatigued uterus. The generated uterine contraction features can then be combined to generate labor features using dropout and average pooling over a window of P samples, followed by adaptive max pooling to produce a single value for each uterine contraction feature input. These labor features effectively collapse each sequence of uterine contraction features into a single labor feature that performs low-pass filtering to improve the signal-to-noise ratio. The result is a state vector of J values (each corresponding to the largest local average value of a single uterine contraction feature) that represents the state of labor.

Some women or birthing people will have a state vector with a signal corresponding to only non-fatigue scenarios (i.e., unlikely to have PPH), others will have signals corresponding to fatigue scenarios (i.e., likely to have a PPH). Assuming monitoring begins during active labor, most women or birthing people will have a mix of the signals corresponding to an initially non-fatigued uterus that transitions to a fatigued uterus as labor continues. Consequently, to produce an estimate for PPH risk, dropout and a linear predictor can be utilized to discriminate women or birthing people who are likely to need an intervention for PPH from those who won't.

Referring back to FIG. 1, the signal and system monitoring component outputs as well as the PPH risk predictor (“VasowatchAl”) algorithm outputs (as outlined in FIG. 2 and described above) can be transmitted to a clinical decision support feedback system. The purpose of this system is to alert clinicians when a patient transitions from low-risk to high-risk and to provide system status feedback via a dashboard. When a subject transitions from low-risk to high-risk a notification can be sent via an alerting mechanism to the clinician of record. Simultaneously, the dashboard at the bedside/nurses station can indicate that the patient is now at high-risk.

In addition, any received signal and system monitoring information can also be displayed on the dashboard and sent to the clinician of record as an alert as appropriate Moreover, the clinical decision support feedback component can have local storage to retain patient state and monitor for extended periods of time where no information is received from the computing component (likely indicating network failure at some junction). This can be imperative in scenarios where the bedside monitor/wearable, computing and database, and clinical feedback components are not located on the same medical device (i.e., involves some amount of non-local networking—likely involving a hospital intranet and/or internet).

Upon receiving alerts and notifications from the system described above, clinical staff can consider the holistic patient and make a suitable determination for course of treatment consistent with protocols for clinical standards-of-care. The system described above is adjunctive in nature and can provide actionable information to the clinician in the minutes and hours leading up to delivery and a potential postpartum hemorrhage.

### Materials and Methods

A primary, prospective, observational study comprising PPG data from patients admitted to a single academic hospital was performed to derive and validate the PPH risk prediction algorithm. The derivation and validation were performed by the same research team and approved by the Institutional Review Board (IRB) of the University. All participants provided written, informed consent before enrollment.

Study Population. Participants were recruited from the obstetric and midwifery practices of patients who presented to a single tertiary center to give birth. Parturients 18 years of age or older were eligible to enter the study if admitted to give birth to a term (≥37 weeks gestation) live, singleton infant. In this study, parturients were excluded if they were preterm (<37 weeks gestation), had a fetal demise, or were pregnant with multiples (twins, triplets, etc.). Additionally, those admitted for planned cesarean births were omitted from the study population since they were presumed to not be experiencing contractions and would not be at risk for developing myometrial fatigue (the hypothesized method of action). Those with less than one hour of PPG data prior to delivery were removed from the final cohort and the remaining women were randomized to derivation and validation cohorts prior to algorithm development, where the validation cohort was blinded during algorithm development.

In the study population, since myometrial fatigue was not directly measurable, administration of nonprophylactic uterotonics was recorded as a reference standard and subjects were retrospectively assigned to case and control populations after all PPG data collection based on the use (case) or absence (control) of nonprophylactic uterotonics. The use of nonprophylactic uterotonics as a reference standard aligns with the aim to provide a real-time PPH risk assessment during active labor since their use is the initial response to any perceived PPH after delivery. Admission PPH risk score was recorded for each subject as a baseline.

Monitoring. Participants wore a commercially available Samsung© Galaxy Watch Active to collect PPG data. An app collected PPG data (e.g., Raproto, Philadelphia, PA), which was transmitted via Wi-Fi using a data transfer protocol called message queueing telemetry transport quality of service 1, which ensures that every data point is received and then stored on a cloud-based platform (e.g., Thingsboard, New York, NY). The expected battery life of this device was 18 to 24 hours. When the remaining battery life reached less than 20%, the clinical study team replaced the low battery watch with a fully charged watch and recorded the time and watch identifier. During the study, patients and clinical staff were told that the watches could be removed at any time if they were uncomfortable, interfered with clinical treatment, or for any other reason they chose. To ensure conditions were representative of real-world practice, no instructions to change clinical procedures were given while the patient was being monitored. No PPG data was revealed to the subjects or clinical care team, thus there was no opportunity to influence the patient care setting.

Algorithm Derivation. While the goal was to create an algorithm that recognizes PPG waveform signatures corresponding with myometrial fatigue, this goal is confounded by the fact that myometrial fatigue was not directly measurable, meaning a uterine biopsy was unobtainable and an intrauterine pressure catheter was not employed. Consequently, the algorithm was derived leveraging the use of nonprophylactic uterotonics as a surrogate reference standard and using a parameter-invariant method designed to maximize diagnostic performance and generalizability in the presence of confounding factors and surrogate reference standards.

This approach has been previously used to develop multiple medical classifier algorithms requiring high sensitivity and specificity along with stable performance across patients without outliers.

The parameter-invariant method uses a statistical first-principles approach to derive algorithms that are invariant to patient-specific parameters (e.g., skin color, labor position/movement) as well as system anomalies common in PPG systems (e.g., ambient lighting, sensor bias/drift). As a result, the algorithm achieves stable performance across the population without requiring manual individual tuning. The algorithm derivation methodology is described below. Briefly, using the derivation cohort PPG data, features invariant to patient-specific parameters were identified and a structured convolutional neural network (CNN) then trained leveraging adaptive max pooling, combining the features to maximize stability and accuracy for discriminating the use of nonprophylactic uterotonics. Using CNNs with adaptive max pooling is a common technique when enforcing monotonic predictions based on time-series data signatures. As more signatures are observed as present in a parturient, the likelihood of receiving nonprophylactic uterotonics increases. Of note, this monotonicity is consistent with the desire to maximally align the predictions with increasing myometrial fatigue. An open-source implementation of the described algorithm is available for academic and noncommercial use (https://james weimer.net/VasowatchAl/).

Data pre-processing. To design a wearable device for predicting PPH risk, only photoplethysmography (PPG) data was utilized in the PPH risk prediction analysis. While incorporating additional sensors, such as electronic health record data and nurse inputs, would theoretically enable higher levels of personalization, they would also increase dependencies on future costly integrations and human input. Consequently, this work aimed to utilize off-the shelf low-power PPG-based devices (e.g., smart watches) to predict PPH risk and the pre-processing considered herein assumed only PPG data were available.

PPG data common in optical wrist-worn devices produced, at time k, optical reflectance data, p(k)∈P, but were also susceptible to bias due to skin color and ambient light. As a consequence, the transformation x̅_{K}: P^{K}→X(K) is applied, where X(K) corresponds to the robust feature space of all sequences of peak-to-peak intervals based on K PPG samples. In practice, this space and corresponding transformation can be calculated using standard techniques (e.g., findmax functions). For notational simplicity, we denote x̅_{K}(p(0), . . . p(K)) as xₖ∈X(k). The intuition for using only the peak-to-peak intervals as features is that while skin color and ambient light almost certainly affect the magnitude of the PPG signal, they do not affect maternal heart beat-to-beat timing. This pre-processing step served to eliminate inherent system biases that are likely to occur during real-world deployments and are consistent with other data pre-processing techniques for PPG data without access to contextual information, such as race and ambient lighting conditions.

Test statistic engineering. To engineer a test statistic for discriminating individuals needing intervention for PPH, start by identifying nuisances that confound the PPG-based maternal heart beat-to-beat timing signal. Begin with intra-subject nuisances (i.e., confounding factors that vary with a single individual). First, observe that phase shifts in maternal heart beat-to-beat timing (i.e. maternal heart rate) are confounded by the PPG sampling rate and when the PPH risk prediction (or predictor) algorithm is executed—simply stated, we don't want the algorithm to produce different answers based on small delays in sampling and/or algorithm execution. Second, the variance of maternal heart beat-to-beat timing (i.e., maternal heart beat-to-beat variability) is confounded by the sympathetic and parasympathetic nervous system and humoral factors which likely dominates any effects of maternal heart beat-to-beat variability due to myometrial fatigue. The effect of these nuisances was modeled on the maternal heart beat-to-beat timing signal as a set of endomorphisms,

$${G}_{K}^{0} ,$$

over the domain of the maternal heart beat-to-beat timing signals, X(K), namely

$${G}_{K}^{o} = \{ \begin{matrix}\begin{matrix}g : {x}_{k} ∈ X ( k ) ↦ \\ ( \begin{matrix}\frac{1}{K} {1}_{K} {1}_{K}^{T} + {U}_{K} {U}_{K - 1} ( \frac{1}{K - 2} {1}_{k - 2} {1}_{k - 2}^{T} + {c}_{2} {U}_{k - 2} R {U}_{k - 2}^{T} ) \\ {U}_{k - 2}^{T} {U}_{K}^{T}\end{matrix} ) {x}_{K} + {U}_{K} {1}_{K - 1} {c}_{1} ,\end{matrix} \\ {U}_{K} {U}_{K}^{T} = I - \frac{1}{K} {1}_{K} {1}_{K}^{T} , {R}^{T} = {R}^{- 1} , ∀ {c}_{1} , {c}_{2} ∈ ℝ\end{matrix} \}$$

where g∈G_{K} denotes a potential heart beat-to-beat timing covariate shift. Consequently, a test statistic was sought that can assess PPH risk robust to heart beat-to-beat timing covariate shift.

A promising approach to realize a robust test statistic utilizes parameter invariant (PAIN) statistics—which have been previously applied in multiple domains. Given a group of nuisance transformations, a PAIN statistic, t, seeks to provide invariance to the nuisance transformations (i.e., is invariant: ∀x∈Xₖ, ∀g∈Gₖ, tₖ(g(x))=tₖ(x)) while only eliminating information affected by the nuisance transformations, (i.e., is maximal ∀x, x′∈Xₖ, ∃g∈Gₖ, tₖ(x)=tₖ(x′)→g(x)=x′). Thus, a candidate was considered PAIN statistic,

$${t}_{k} \text{:} x ∈ {X}_{k} ↦ ( \frac{1}{\sqrt{k}} {1}_{k}^{T} {x}_{,} \frac{1}{\sqrt{k - 2}} {1}_{k - 2}^{T} {U}_{k - 1}^{T} {U}_{k}^{T} x ) ∈ {ℝ}^{2}$$

and proved it to be invariant since, ∀x∈Xₖ, ∀g∈Gₖ:

$$t ( g ( x ) ) = ( \frac{1}{\sqrt{k}} {1}_{k}^{T} g ( x ) , \frac{1}{\sqrt{k - 2}} {1}_{k - 2}^{T} {U}_{k - 1}^{T} {U}_{k}^{T} g ( x ) ) = ( \frac{1}{\sqrt{k}} {1}_{k}^{T} x , \frac{1}{\sqrt{k - 2}} {1}_{k - 2}^{T} {U}_{k - 1}^{T} {U}_{k}^{T} ( x + {1}_{k} {c}_{1} + {U}_{k} {1}_{k - 1} {c}_{2} ) ) = ( \frac{1}{\sqrt{k}} {1}_{k}^{T} x , \frac{1}{\sqrt{k - 2}} {1}_{k - 2}^{T} {U}_{k - 1}^{T} ( {U}_{k}^{T} x + {1}_{k - 1} {c}_{1} ) ) = ( \frac{1}{\sqrt{k}} {1}_{k}^{T} x , \frac{1}{\sqrt{k - 2}} {1}_{k - 2}^{T} {U}_{k - 1}^{T} {U}_{k}^{T} x ) = t ( x )$$

and maximal since, ∀x, x′∈Xₖ, ∃g∈Gₖ,

$$t ( x ) = t ( {x}^{′} )$$

$$\to ( {1}_{k}^{T} x = {1}_{k}^{T} {x}^{′} , {1}_{k - 2}^{T} {U}_{k - 1}^{T} {U}_{k}^{T} x = {1}_{k - 2}^{T} {U}_{k - 1}^{T} {U}_{k}^{T} {x}^{′} )$$

$$\to ( {1}_{k}^{T} ( x - {x}^{′} ) = 0 , {1}_{k - 2}^{T} {U}_{k - 1}^{T} {U}_{k}^{T} ( x - {x}^{′} ) = 0 )$$

$$\to ∃ {c}_{1} , {c}_{2} ∈ ℝ , ∃ R ∈ \{ R \vert {R}^{T} = {R}^{- 1} \} ,$$

$${U}_{K} {U}_{K - 1} ( \frac{1}{K - 2} {1}_{k - 2} {1}_{k - 2}^{T} + {c}_{2} {U}_{k - 2} R {U}_{k - 2}^{T} ) {U}_{K - 1}^{T} {U}_{K}^{T} x + {1}_{K} {c}_{0} + {U}_{K} {1}_{K - 1} {c}_{1} = {x}^{′}$$

$$\to ∃ g ∈ {G}_{k} , g ( x ) = {x}^{′}$$

As a final step, at time k, concatenate the test statistics from time 0 to k and write sₖ=(t₀(x₀), . . . , tₖ(xₖ)) to be the time-series evolution of the test statistics for an individual subject.

Model Architecture. The model architecture of FIG. 2 was utilized for the PPH risk predictor using pulse waveform data. The only trainable parameters in the model were contained in the convolution layer and linear output layer. The other layers were intended to smooth out the predictions such that the resulting model is robust and generalizable.

Model Training. The model architecture was trained on the derivation cohort (n=187). The model contained five parameters, two in the convolutional layer, and three in the linear layer. While a larger number of parameters in the convolutional and linear layers could have been utilized, to maximize robustness and generalizability the model was purposely constrained to five parameters. To improve training performance, the derivation cohort was reduced when training the initial model to include only subjects satisfying at least one of the following:
No administration of nonprophylactic uterotonics and an estimated blood loss below 250 mL for vaginal delivery and 500 mL for cesarean delivery.
Administration of a single nonprophylactic uterotonic and an estimated blood loss of over 500 mL for vaginal delivery or 1000 mL for cesarean delivery.
Administration of multiple nonprophylactic uterotonics.

These thresholds were chosen to such that the labeling of subjects as controls (not receiving nonprophylactic uterotonics) and cases (receiving nonprophylactic uterotonics) strongly correlated with the need for nonprophylactic uterotonics. As a result, the reduced derivation cohort contained 33 women who received nonprophylactic uterotonics (case subjects) and 29 women who did not (control subjects). Even with this reduced deviation cohort, there was ample data to initially train the five parameter model: 62 subjects/5 parameters=12.4 subjects per parameter—where a general rule of thumb (but not a requirement) for promoting robustly trained models is to have at least 10 independent subjects per parameter. Once the initial model was trained, the entire derivation cohort was re-introduced to optimize the decision threshold for population performance. The model derived following the process described herein was validated.

Algorithm Integration into clinical care. An example architecture of how the PPH risk prediction algorithm could be implemented into clinical care settings is pictured in FIG. 1. The architecture includes bedside and/or wearable medical devices that produce pulse waveform information. While this paper focuses on PPG data, ballistography (accelerometry data) and electrocardiogram (ECG data) also contain heart beat-to-beat timing information. Consequently, the PPH risk prediction (or predictor) algorithm derived and validated herein could extend to a wealth of other medical monitoring system—not just wearable producing PPG data.

The heart beat-to-beat timing signal can be transmitted over a wired/wireless network and integrated with computing and database storage, including hospital electronic medical records. The PPH risk predictor algorithm can be run on a fixed schedule (e.g., 5 minutes) to update PPH risk prediction via a clinical decision support feedback system consisting of a nurse station dashboard and/or bedside monitors. The proposed architecture generalizes to both cloud-based computing scenarios (e.g., for large volume systems) as well as wearable/bedside computing scenarios (e.g., for remote settings).

Validation. The final candidate algorithm was validated using an independent and blinded test data set. For this preplanned analysis, the algorithm evaluated individual patient data and was executed every 5 minutes during active labor. The performance of the population was then calculated using medians and interquartile ranges of the performance. As a result of using message queueing telemetry transport quality of service 1 to transfer data to the cloud, the accelerometry data set had no missing data. However, it remains possible that data will be missing, at least temporarily, in a real-world implementation of a clinical PPH risk predictor, in which case the algorithm can be designed to handle data in the following manner: Data can be timestamped by the PPG devices and, when evaluated by the PPH risk prediction algorithm, missing data can be treated as missing and not imputed. Examples of how missing data may occur in a real-world implementation include a weak or erratic Wi-Fi/Bluetooth network, which would lead to temporary delays in communication of data, although it would eventually be collected and analyzed when the Wi-Fi signal allows. Alternatively, if a device runs out of power, PPH risk prediction will not be possible during that time because PPG data cannot be collected.

Statistical Analysis. The PPH risk prediction algorithm performance was evaluated in terms of sensitivity, positive predictive value, and prediction timing. For sensitivity, the percentage of women or birthing people receiving nonprophylactic uterotonics was reported that are also identified by the algorithm as being high-risk for PPH. For positive predictive value, the percentage of women or birthing people the algorithm predicts as high-risk was reported that also received nonprophylactic uterotonics. For prediction timing, the time prior to delivery was recorded that the algorithm predicts a woman/women or birthing person/people is/are high risk. Note, that since the algorithm (as described above) is designed to have monotonic risk predictions, once a woman or birthing person is identified as high-risk by the algorithm, the algorithm will continue to identify the woman or birthing person as high risk until the device is removed after delivery. Consequently, the prediction timing analysis is based on the earliest time a woman or birthing person is predicted as high-risk. For all performance metrics, confidence interval estimates were reported based on the number of women or birthing people used in the calculation of the performance metric. Finally, whether patient-specific factors would lead to variations in performance of the algorithm were evaluated by comparing the sensitivity, positive predictive value, and prediction timing by age, gestation, race, ethnicity, birth type, and the use of anesthesia using Wilcoxon rank sum testing.

Sample Size. The target enrollment of 200 subjects in the derivation cohort was based on prior work on parameter invariant algorithms, which suggested that at least 1500 hours of PPG data was needed prior to delivery to derive the PPH risk prediction algorithm. The validation cohort sample size was not based on a sample size calculation and instead was determined to be approximately the same number of patients as the derivation cohort.

### Results

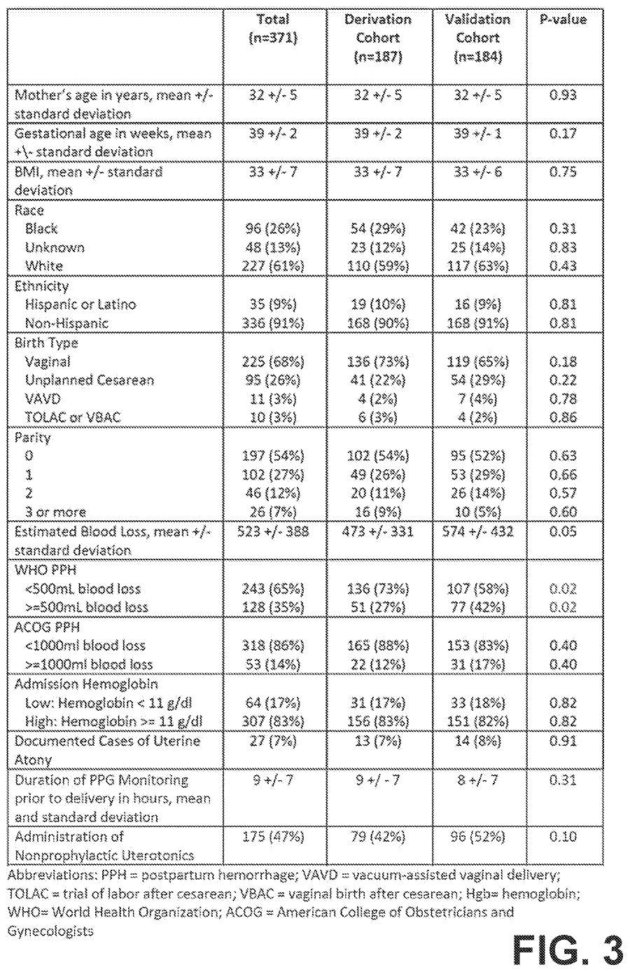

From Jun. 4, 2021, through Sep. 28, 2021, 371 patients, including 187 in the derivation cohort and 184 in the validation cohort, were enrolled. The algorithm derivation cohort included 79 case subjects receiving nonprophylactic uterotonics and 108 control subjects. In total, 1683 hours of PPG data prior to delivery were acquired, with a mean 9 hours per subject for algorithm derivation. The algorithm validation cohort included 96 case subjects receiving nonprophylactic uterotonics and 88 controls, totaling 1472 hours of PPG data prior to delivery with a mean of 8 hours per subject. The table of FIG. 3 presents the clinical and demographic characteristics of the derivation and validation cohorts. While most characteristics were not statistically significant, subjects in the validation cohort experienced higher mean blood loss (574 mL vs. 473 mL, p-value=0.05) when compared to the derivation cohort. Moreover, the number of PPHs in each cohort was significantly different when comparing the current American College of Obstetricians and Gynecologists (ACOG) standard vs. the World Health Organization (WHO) standard (51 vs. 71 PPH events, p-value=0.02). The administration of non-prophylactic uterotonics was similar in the derivation and validation cohorts (52% vs 42%, p-value=0.10), as were documented cases of uterine atony (13 versus 14, P=0.91). For both the algorithm derivation and validation cohorts, the watches were well tolerated. Less than 5% of the patients in the derivation and the validation cohorts removed the devices and prematurely terminated the study prior to delivery. Nurses reported no issues with the wrist-worn watches interfering with clinical care.

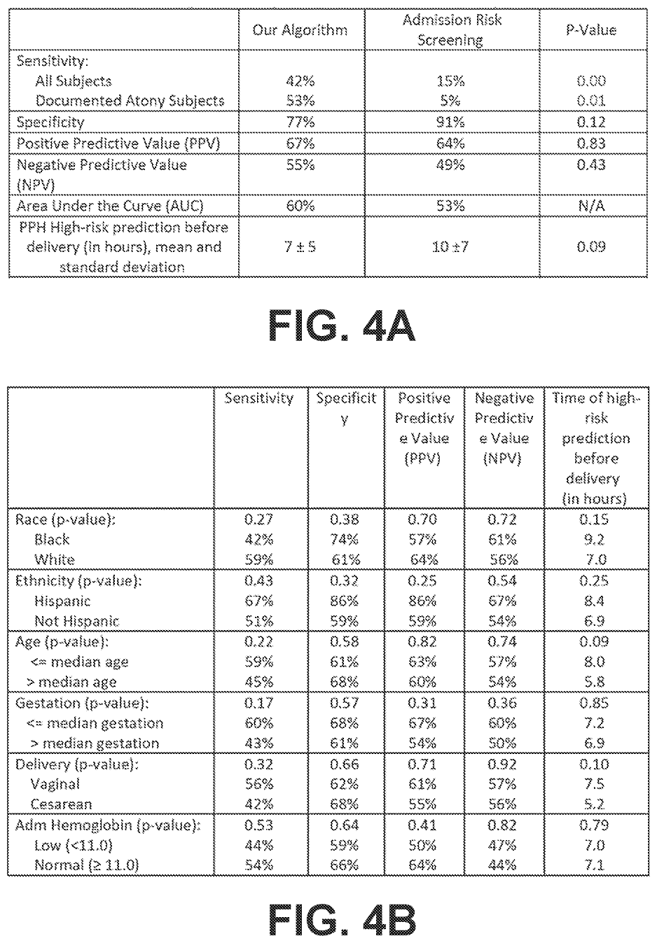

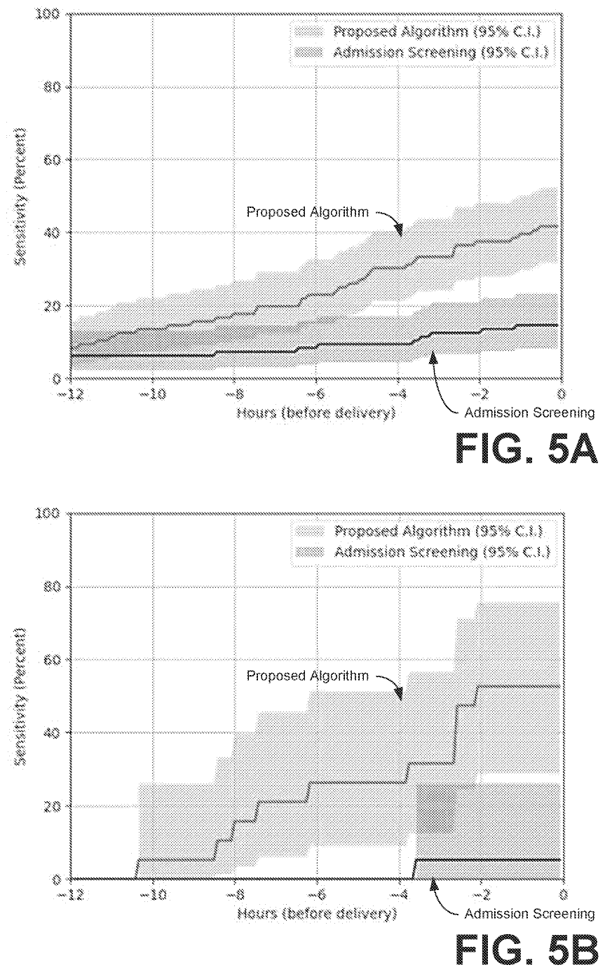

Algorithm Performance. The table of FIG. 4A reports the performance of the PPH risk prediction (or predictor) algorithm leveraging a PPH admission risk prediction baseline for comparison. In the table of FIG. 4A, the sensitivity is significantly different when analyzing the entire validation cohort (42% vs. 15%, p-value<0.01) and when only considering the validation subgroup with documented uterine atony (53% vs. 5%, p-value<0.01). Other than sensitivity, there were no significant differences observed between specificity, positive predictive value, negative predictive value, or in terms of high-risk prediction lead-time, as reported in the table of FIG. 4A. Expanding upon the sensitivity results, FIGS. 5A and 5B display the average and 95% confidence interval for the percentage of cases that are predicted to be at high-risk for PPH (i.e., sensitivity) against the time before delivery in hours. FIG. 5A illustrates the pre-birth sensitivity of the PPH risk prediction algorithm compared with admission risk scoring on the validation cohort and FIG. 5B illustrates the pre-birth sensitivity of the PPH risk prediction algorithm compared with admission risk scoring on parturients with documented uterine atony on the validation cohort. These results are presented in terms of all test cases receiving nonprophylactic uterotonics (FIG. 5A) and restricted to the subset of women with documented uterine atony (FIG. 5B).

The performance of the proposed PPH risk prediction algorithm and the standard admission risk screening are shown for comparison in FIGS. 5A and 5B. Both approaches in both scenarios exhibit increasing sensitivity as time approaches delivery time, where the cause of increasing performance of the admission risk screening approach is a direct consequence of the time when women or birthing people were admitted to the hospital. The sensitivity of the proposed PPH risk prediction algorithm has an estimated average that is significantly better than admission risk scoring starting about 6 hours prior to delivery and reaches a peak performance differential of nearly 3× better than admission risk screening at time of delivery (42% vs. 15%,) nearly 3× better than the admission risk criteria at 6 hours prior to delivery (38% vs. 13%) and nearly 3× better at time of delivery (42% vs. 15%). When considering only documented uterine atony cases, the proposed PPH risk prediction algorithm has an estimated average that is over 10× better at 2 hours prior to delivery (53% vs. 5%). The difference in sensitivity on documented cases of uterine atony between the proposed PPH risk prediction approach and the baseline comparison was statistically significant (p-value<0.05) beginning at 6 hours prior to delivery.

Importantly, the PPH risk prediction algorithm was unaffected by confounding demographic characteristics representing patient-specific factors that could theoretically lead to variable performance. Most notably, and as reported in the table of FIG. 4A, there were no significant differences in sensitivity, specificity, positive predictive value, or negative predictive value based on race (Black vs. white), ethnicity (Hispanic versus non-Hispanic), age (older versus younger than the cohort median), gestation (longer versus shorter than the cohort median), delivery type (vaginal vs. cesarean). The area under the receiver operator characteristic (ROC) curve, also known as the area under the curve (AUC), is larger for the proposed algorithm when compared against the admission risk prediction approach (60% versus 53%). The table of FIG. 4B illustrates the significance of the PPH risk prediction algorithm performance variability on validation cohort.

Principle Findings. Postpartum hemorrhage risk prediction using a patient's own PPG data can significantly improve prediction performance prior to delivery in terms of sensitivity and positive predictive value when compared to current admission PPH risk screening tools. Statistically significant improvements can be observed at 6 hours prior to delivery. Moreover, PPH prediction using PPG data can be robust to patient-specific confounding factors such as race, ethnicity, age, and gestation.

Results in the Context of What is Known. The performance of the PPH risk prediction algorithm is significantly different than other proposed techniques for predicting PPH. This is due to the fact that the literature focusing on PPH prediction is largely concerned with predicting (severe) PPH, not the use of nonprophylactic uterotonics. These are fundamentally different problems since predicting PPH must account for the effect of routine clinical care (i.e., the administration of uterotonics) which may make estimating PPH a more challenging problem. PPG data is known to have significant variability based on the monitoring subject's skin color. However, the results suggest that the performance of the proposed PPH risk prediction algorithm is robust without regard to skin color. This is a direct consequence of the parameter-invariant approach (detailed above) that aims to be maximally invariant to skin color by ignoring the magnitude of the PPG waveform and only monitors the beat-to-beat timing (as indicated by the estimated R-R interval).

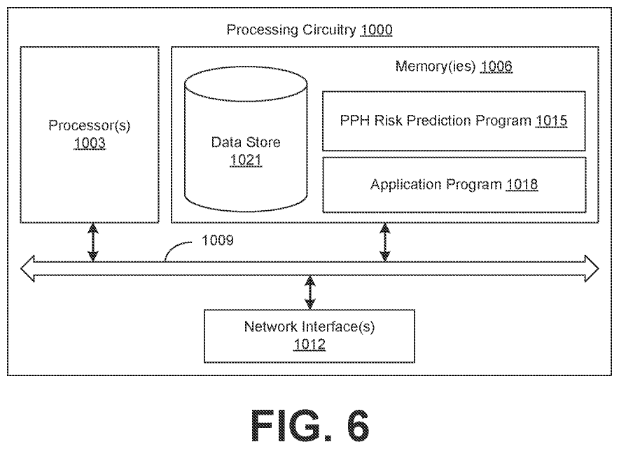

Referring next to FIG. 6, shown is a schematic diagram illustrating an example of a processing circuitry 1000 that can be used for PPH risk prediction methodology as described, in accordance with various embodiments of the present disclosure. The processing circuitry 1000 can include at least one processor circuit having, for example, a processor 1003 and a memory 1006, both of which are coupled to a local interface 1009. The local interface 1009 may comprise, for example, a data bus with an accompanying address/control bus or other bus structure as can be appreciated. The processing circuitry 1000 can comprise one or more computing/processing device such as, e.g., a smartphone, tablet, computer, controller, etc. To this end, each processing circuitry 1000 may comprise, for example, at least one server computer or like device, which can be utilized in a cloud-based environment.

In some embodiments, the processing circuitry 1000 can include one or more network interfaces 1012. The network interface 1012 may comprise, for example, a wireless transmitter, a wireless transceiver, and/or a wireless receiver. The network interface 1012 can communicate to a remote computing/processing device or other components using a Bluetooth, WiFi, or other appropriate wireless protocol. As one skilled in the art can appreciate, other wireless protocols may be used in the various embodiments of the present disclosure. The network interface 1012 can also be configured for communications through wired connections.

Stored in the memory 1006 are both data and several components that are executable by the processor(s) 1003. In particular, stored in the memory 1006 and executable by the processor 1003 can be a PPH risk prediction application 1015 which can control power converter operation as disclosed herein, and potentially other applications 1018. In this respect, the term “executable” means a program file that is in a form that can ultimately be run by the processor(s) 1003. Also stored in the memory 1006 may be a data store 1021 and other data. In addition, an operating system may be stored in the memory 1006 and executable by the processor(s) 1003. It is understood that there may be other applications that are stored in the memory 1006 and are executable by the processor(s) 1003 as can be appreciated.

Examples of executable programs may be, for example, a compiled program that can be translated into machine code in a format that can be loaded into a random access portion of the memory 1006 and run by the processor(s) 1003, source code that may be expressed in proper format such as object code that is capable of being loaded into a random access portion of the memory 1006 and executed by the processor(s) 1003, or source code that may be interpreted by another executable program to generate instructions in a random access portion of the memory 1006 to be executed by the processor(s) 1003, etc. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores, and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, ultrasound or other devices. The processor 1003 may be of electrical or of some other available construction.

Although the PPH risk prediction application 1015, and other various applications 1018 described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the PPH risk prediction application 1015, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the PPH risk prediction application 1015, may be implemented and structured in a variety of ways. For example, one or more applications described may be implemented as modules or components of a single application. For example, the PPH risk prediction application 1015 can include a wide range of modules such as, e.g., an initial model or other modules that can provide specific functionality for the disclosed methodology. Further, one or more applications described herein may be executed in shared or separate computing/processing devices or a combination thereof. For example, a plurality of the applications described herein may execute in the same processing circuitry 1000, or in multiple computing/processing devices in the same computing environment. To this end, each processing circuitry 1000 may comprise, for example, at least one server computer or like device, which can be utilized in a cloud-based environment.

Conclusion. A PPH risk prediction (or predictor) algorithm was developed using non-invasive photoplethysmography (PPG) data to provide intrapartum decision support indicating subjects at high-risk for requiring postpartum nonprophylactic intervention for hemorrhage. The PPH risk prediction algorithm was designed using machine learning to constrain the learning process such that the resulting model targets myometrial fatigue for predicting need for PPH intervention. A wearable photoplethysmography (PPG) device was placed on a participant's wrist from active labor through delivery and 24 hours postpartum that collected maternal pulse waveform data. The PPH risk prediction algorithm was designed to recognize PPG waveform signatures that corresponded to specific variations in maternal heart beat-to-beat timing modeled to correlate with myometrial fatigue.

Elevated risk for PPH intervention was identified 1 to 6 hours ahead of delivery with 3-10 times higher sensitivity than admission PPH risk scores, regardless of skin color and other factors. More accurate advanced warning of the need for probable PPH intervention would allow time to mobilize resources for appropriate and timely treatment. Patient care and resources could be better allocated to ensure high-risk subjects receive additional attention during active labor and birth. Racial inequities currently exist in PPH identification and treatment. This risk-identifying technology has the potential to reduce current racial inequities in PPH risk identification and treatment and offers the most innovative approach in decades.

A software-as-a-medical device system can non-invasively predict a PPH more accurately than the state-of-the-art prior to delivery. There is a significant effort to provide early prediction to enable proactive clinical planning. Current solutions rely on PPH detection rather than PPH prediction. Where PPH detection approaches relying on traditional methods, such as blood pressure and pulse rate indicators, often yield vital information too late, and relying on checking sanitary pads after birth, which is old-fashioned and dangerous. Current solutions only work towards what to do after it has been detected, which may often be too late, resulting in death or significant morbidity. The proposed PPH risk prediction methodology leverages maternal heart beat-to-beat information collected by several existing devices in the labor and delivery unit. The system can create a new vital sign that provides a surrogate measure of uterine fatigue that is suspected of being a strong predictor of uterine atony—which accounts for over 80% of all PPH. This vital sign can utilize waveform data collected at >5 Hz from a wearable or minimally invasive monitoring device (e.g., pulse oximeter, ECG, maternal fetal monitor). The device can perform local pre-processing and filtering of the data. In coordination with a cloud-based backend, the wearable device and algorithms in the cloud can further process the data to predict whether a life-threatening postpartum hemorrhage is likely to occur. An alerting mechanism integrated with the hospital EHR can notify clinical staff to the situation.

The use of the above device has multiple benefits that can be reaped, such as:
Significant hospital and insurance costs savings associated with morbidity and mortality;
Saving the lives of mothers, especially Black and brown women or birthing people, who are dying at rates 2-4× greater than white women or birthing people;
The ability to work with devices that will measure multiple vital signs wirelessly; and
To be another set of eyes and ears for the health care providers to alert them to a hemorrhage.

The current performance of the proposed system (based on a 525 patient IRB-approved observational study) is 60%-80% sensitivity at correctly predicting PPH interventions with a positive predictive value (PPV) >50%. For comparison, the current state-of-the-art based on admission risk criteria for predicting PPH provides a 20% sensitivity and 50% PPV. Thus, with minimal/no hardware changes to the current labor and delivery unit landscape, and with no decrease in positive predictive value, the proposed system will catch at least 3× more PPH that the current state-of-the-art.

Currently, an insurer pays a global fee for a vaginal birth and a slightly higher fee for a cesarean birth. The hospital incurs many expenses related to medications, blood transfusions, labor costs, additional operating room time, supplies and more when a hemorrhage occurs. It is expected that the proposed PPH risk prediction device can save a hospital money, save lives and decrease morbidity. The reimbursement model may not change but there may be an interest in insurance companies to support and pay for the device separate from the global fee they currently pay. In addition, global health NGO's and government insurers may be very interested in the product to save lives, in order to greatly expand their maternal care work and efforts currently in place to support mothers in childbirth and maternal health care, in general.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A postpartum hemorrhage (PPH) risk prediction system, comprising:
one or more pulse waveform sensors configured to collect heart beat-to-beat timing information of an individual in real time; and
processing circuitry configured to:
process the heart beat-to-beat timing information to monitor for previously learned signatures predictive of uterine atony risk; and
provide a clinical alert notification in response to the monitoring, the notification provided through an audible, haptic, dashboard or mobile device.

2. The PPH risk prediction system of claim 1, wherein the heart beat-to-beat timing information is collected from photoplethsymgraphy, electrocardiogram, ballistography, or electromyarthapy.

3. The PPH risk prediction system of claim 1, wherein a bed-side or wearable medical device comprises the one or more pulse waveform sensors.

4. The PPH risk prediction system of claim 3, wherein the wearable medical device is a smart watch.

5. The PPH risk prediction system of claim 3, wherein the collected heart beat-to-beat timing information is stored in memory on the wearable medical device.

6. The PPH risk prediction system of claim 1, wherein the processing circuitry is locally or remotely located.

7. The PPH risk prediction system of claim 6, wherein the remotely located processing circuitry comprises at least one server computer in a cloud-based environment.

8. The PPH risk prediction system of claim 7, wherein the remotely located processing circuitry receives the collected heart beat-to-beat timing information from the one or more pulse waveform sensors.

9. The PPH risk prediction system of claim 8, wherein the collected heart beat-to-beat timing information is stored in memory on the at least one server computer.

10. A method for postpartum hemorrhage (PPH) risk prediction, comprising:
obtaining, via one or more pulse waveform sensors, heart beat-to-beat timing information of an individual in real time;
processing, by processing circuitry, the heart beat-to-beat timing information to monitor for previously learned signatures predictive of uterine atony risk; and
providing, by the processing circuitry, a clinical alert notification in response to the monitoring, the notification provided through an audible, haptic, dashboard or mobile device.

11. The method of claim 10, wherein the heart beat-to-beat timing information is collected from photoplethsymgraphy, electrocardiogram, ballistography, or electromyarthapy.

12. The method of claim 10, wherein the heart beat-to-beat timing information is collected via a bed-side or wearable medical device comprising the one or more pulse waveform sensors.

13. The method of claim 12, wherein the wearable medical device is a smart watch.

14. The method of claim 10, wherein the heart beat-to-beat timing information is communicated from the one or more pulse waveform sensors to the processing circuitry for processing.

15. The method of claim 14, wherein the heart beat-to-beat timing information is communicated to the processing circuitry via a wireless connection.

16. The method of claim 14, wherein the processing circuitry is remotely located.

17. The method of claim 16, wherein the remotely located processing circuitry comprises at least one server computer in a cloud-based environment.

18. The method of claim 17, wherein the communicated heart beat-to-beat timing information is stored in memory on the at least one server computer.
